(19) United States
(12) Patent Application Publication
(10) Pub. No.: US 2010/0099118 A1
(43) Pub. Date: Apr. 22, 2010

(54) METHODS OF DETERMINING TOTAL PON1 LEVEL

(75) Inventors: Leonid Gaydukov, Moscow (RU); Olga Khersonsky, Jerusalem (IL); Dan S. Tawfik, Jerusalem (IL)

Correspondence Address: MARTIN D. MOYNIHAN d/b/a PRTSI, INC., P.O. BOX 16446, ARLINGTON, VA, 22215, US

(73) Assignee: Yeda Research And Development Co., Ltd., Rehovot (IL)

(21) Appl. No.: 12/450,167

(22) PCT Filed: Mar. 11, 2008
(86) PCT No.: PCT/IL2008/000335

§ 371 Date: Sep. 14, 2009

Related U.S. Application Data
(60) Provisional application No. 60/918,063, filed on Mar. 15, 2007.

(51) Int. Cl. G01N 33/573; C07F 9/06

(52) U.S. Cl. 435/7.4; 549/222

(57) ABSTRACT

A method of determining an amount of total PON1 in a sample of a subject is disclosed. The method comprises:
(a) contacting the sample with a compound being capable of generating at least one spectrophotometrically detectable moiety upon contact with PON1, under conditions wherein the generating is not dependent on a PON1 status; and
(b) spectrophotometrically measuring a level of the moiety, thereby determining an amount of total PON1 in the sample. Kits for measuring total PON1 levels comprising the compounds are also disclosed.

### FIELD AND BACKGROUND OF THE INVENTION

The present invention relates to a method of measuring total PON1 levels in serum and, more particularly, to a chromogenic fluorogenic phosphotriester substrate (DEPCyMC) for the enzymatic assays of total PON1 levels irrespective of its HDL status and polymorphism.

Atherosclerosis is a disorder characterized by cellular changes in the arterial intima and the formation of arterial plaques containing intracellular and extracellular deposits of lipids. The thickening of artery walls and the narrowing of the arterial lumen underlies the pathologic condition in most cases of coronary artery disease, aortic aneurysm, peripheral vascular disease, and stroke. A number of metabolic pathways and a cascade of molecular events are involved in the cellular morphogenesis, proliferation, and cellular migration that results in atherogenesis (Libby et al. (1997) Int J Cardiol 62 (S2):23-29).

Serum paraoxonase (PON1) is a high density lipoprotein (HDL)-associated enzyme playing an important role in organophosphate detoxification and prevention of atherosclerosis. HDL-bound PON1 inhibits LDL oxidation and stimulates cholesterol efflux from macrophages. PON1 knockout mice are highly susceptible to atherosclerosis. Accordingly, serum PON1 levels seem to be inversely related to the level of cardiovascular disease, although this correlation is weak. PON1 hydrolyzes a broad range of substrates, and has been traditionally described as paraoxonase/arylesterase. However, it recently became apparent that PON1 is in fact a lactonase with lipophylic lactones comprising its primary substrates. Impairing the lactonase activity of PON1, through mutations of its catalytic dyad, diminishes PON1's ability to prevent LDL oxidation and stimulate macrophage cholesterol efflux, indicating that the anti-atherogenic functions of PON1 are likely to be mediated by its lipo-lactonase activity.

PON1 is synthesized in the liver and secreted into the blood where it associates with HDL complexes carrying apolipoprotein A-I (apoA-I). The structural model of PON1 indicates that the HDL surface lies in close proximity to PON1's active site, and thus provides an optimal environment for the enzyme's interaction with its lipophylic substrates. Indeed, it has been shown that PON1 binds HDL-apoA-I particles with nanomolar affinity. HDL-apoA-I binding stabilizes the enzyme and selectively stimulates its lipo-lactonase activity.

The impact of PON1 on atherosclerotic disease and resistance to organophosphate toxicity led to intensive investigations of its natural polymorphisms. These include the 192R/Q which alters PON1's substrate specificity towards organophosphates, M55L, and polymorphisms in the promoter region that affect PON1's expression levels. It has recently been shown that the 192R/Q polymorphs differ in their HDL-binding properties, with the R isozyme exhibiting higher affinity, stability, lipo-lactonase activity, and macrophage cholesterol efflux [Gaidukov, L., M. Rosenblat, M. Aviram, and D. S. Tawfik. 2006. J Lipid Res 47: 2492-2502].

Several studies have concluded that PON1's phenotype, namely the total enzyme levels and activity, are better predictors of the risk of atherosclerotic disease than its genotype [Mackness, M., and B. Mackness. 2004. Free Radic Biol Med 37: 1317-1323; Jarvik, G. P., et al., 2003. Arterioscler Thromb Vasc Biol 23: 1465-1471]. However, to date, blood tests measure phosphotriesterase and arylesterase activities to examine PON1's levels and activity. Because these promiscuous activities of PON1 are hardly stimulated by HDL, and obviously bear no physiological relevance, these tests cannot predict the levels of the PON1-HDL complex, nor its antiatherogenic potential. In addition, these activities are affected by the 192R/Q polymorphism, and thus can only provide a measure of PON1's total levels within the same genotype. While the paraoxonase activity differs significantly between the R/Q polymorphs, the aryl esterase activity of the R polymorph undergoes a 2-fold higher level of catalytic stimulation by HDL [Gaidukov, L., M. Rosenblat, M. Aviram, and D. S. Tawfik. 2006. J Lipid Res 47: 2492-2502].

There is thus a widely recognized need for, and it would be highly advantageous to have, new sera tests that provide a facile and accurate measure of total PON1 levels irrespective of its genotype and HDL status.

### SUMMARY OF THE INVENTION

According to one aspect of the present invention there is provided a method of determining an amount of total PON1 in a sample of a subject, the method comprising:

(a) contacting the sample with a compound being capable of generating at least one spectrophotometrically detectable moiety upon contact with PON1, under conditions wherein the generating is not dependent on a PON1 status; and

(b) spectrophotometrically measuring a level of the moiety, thereby determining an amount of total PON1 in the sample.

According to another aspect of the present invention there is provided a method of determining a normalized enzymatic activity of PON1 in a sample of a subject, the method comprising:

(a) contacting the sample with a compound being capable of generating at least one spectrophotometrically detectable moiety upon contact with PON1, under conditions wherein the generating is not dependent on a PON1 status;

(b) spectrophotometrically measuring a level of the moiety, thereby determining an amount of total PON1 in the sample; and

(c) measuring a PON1 enzymatic activity selected from the group consisting of a lactonase activity, a paraoxonase activity and an aryl esterase activity, wherein a ratio of the PON1 enzymatic activity and the amount of total PON1 is the normalized enzymatic activity of PON1

According to yet another aspect of the present invention there is provided a compound 7-O-Diethylphosphoryl-(3-cyano 4-methyl 7-hydroxycuomarin).

According to still another aspect of the present invention there is provided a kit for diagnosing a disorder associated with abnormal levels or activity of a PON1 in a subject, the kit comprising a phophotriester compound selected from the group consisting of 7-O-Diethyl phosphoryl 3-cyano-7-DDAO, 7-O-diethyl phosphoryl 3-cyano 4-methyl 7-hydroxycoumarin (DEPCyMC) and 7-O-Diethyl phosphoryl 3-cyano-7-hydroxycoumarin (DEPCyC) and instructions for measuring a total amount of PON1.

According to further features in preferred embodiments of the invention described below, the compound is selected from the group consisting of 7-O-Diethyl phosphoryl 3-cyano-7-DDAO, 7-O-diethyl phosphoryl 3-cyano 4-methyl 7-hydroxycoumarin (DEPCyMC) and 7-O-Diethyl phosphoryl 3-cyano-7-hydroxycoumarin (DEPCyC).

According to still further features in the described preferred embodiments the conditions comprise contacting the sample with the compound at pH 9.

According to still further features in the described preferred embodiments measuring the lactonase activity is effected by:

(a) contacting the sample with a compound containing at least one lactone, wherein the compound is capable of generating at least one spectrophotometrically detectable moiety upon hydrolysis of the lactone; and

(b) spectrophotometrically measuring a level of the moiety.

According to still further features in the described preferred embodiments the compound is 5-thiobutyl butyrolactone (TBBL).

According to still further features in the described preferred embodiments the kit further comprises at least one agent for determining in a sample of a subject stability of a serum PON1:HDL apoA-I complex.

According to still further features in the described preferred embodiments the at least one agent for determining a stability of a serum PON1:HDL apoA-I complex is an agent capable of measuring an inactivation rate of an enzymatic activity of a PON1 of said PON1:HDL apoA-I complex.

According to still further features in the described preferred embodiments the at least one agent is a PON1 inactivator.

According to still further features in the described preferred embodiments the PON1 inactivator is NTA, β-mercaptoethanol or both.

According to still further features in the described preferred embodiments the at least one agent is phenyl acetate.

According to still further features in the described preferred embodiments the kit further comprises at least one reagent for determining a lactonase activity of serum PON1.

According to still further features in the described preferred embodiments the at least on reagent is 5-(thiobutyl)-butyrolactone (TBBL).

According to still further features in the described preferred embodiments the disorder associated with abnormal levels or activity of a PON1 is selected from the group consisting of a cardiovascular disorder, a pancreatic disorder and a neurological disorder.

According to still further features in the described preferred embodiments the cardiovascular disorder is selected from the group consisting of atherosclerosis, coronary heart disease, myocardial infarction, peripheral vascular diseases, venous thromboembolism and pulmonary embolism.

The present invention successfully addresses the shortcomings of the presently known configurations by providing a novel method of determining total PON1 levels, irrespective of PON1 status.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A-D are graphs depicting lactonase activity, DEPCyMC activity, lactonase stimulation and fraction of tightly HDL-bound PON1, in human sera from 54 healthy individuals. Horizontal bars represent the mean value for each group.

FIG. 1A depicts levels of lactonase activity, measured with TBBL (0.25 mM) and expressed in Units/ml (1 μmol of TBBL hydrolyzed per min per 1 ml undiluted serum). FIG. 1B depicts total PON1 concentrations, measured with DEPCyMC (1 mM) and expressed in mUnits/ml (1 nmol of DEPCyMC hydrolyzed per min per 1 ml undiluted serum). FIG. 1C depicts stimulation of the lactonase activity, expressed as the ratio of TBBL to DEPCyMC activity for each individual serum. FIG. 1D depicts amplitude of the slow phase of inactivation (A₂, %) which was derived from serum inactivation assay with the calcium chelator NTA and the reducing agent β-mercaptoethanol [Gaidukov, L., M. Rosenblat, M. Aviram, and D. S. Tawfik. 2006. J Lipid Res 47: 2492-2502] (see FIG. 4A), and corresponds to the fraction of tightly HDL-bound PON1.

FIG. 1E is a schematic diagram of the novel substrate of the present invention −7-O-diethyl phosphoryl 3-cyano 4-methyl 7-hydroxycoumarin.

FIG. 2A is a graph depicting a pH rate profile of human PON1 R and Q polymorphs with DEPCyMC. Specific activity of DEPCyMC hydrolysis at 1 mM was measured with the purified human PON1-R and Q polymorphs at the pH range of 7-10. Protein concentrations were verified by enzymatic measurements with phenyl acetate using the reported specific activities of the R and Q polymorphs [Billecke, S., D. 2000 Drug Metab Dispos 28: 1335-1342. Specific activities were expressed in Units (1 unit=1 μmol of DEPCyMC hydrolyzed per minute per 1 mg of protein). Each value represents the mean of three measurements, and the horizontal bars the S.D. of these measurements.

FIG. 2B is a graph depicting the levels of activity with DEPCyMC, phenyl acetate and paraoxon, in human sera from 54 healthy individuals. DEPCyMC activity was measured in 50 mM Bis-trispropane at pH 9.0 and 7.0 with 1 mM CaCl₂. Phenyl acetate and paraoxonase activities were measured in 50 mM Tris pH 8.0 with 1 mM CaCl₂. All activities were measured with 1 mM substrate, and expressed in Units/ml for phenyl acetate (1 μmol of phenyl acetate hydrolyzed per min per 1 ml undiluted serum) or mUnits/ml for other activities (1 nmol of substrate hydrolyzed per min per 1 ml undiluted serum). Horizontal bars represent the mean value for each group.

FIG. 3 is a graph depicting stimulation of the lipo-lactonase activity of the rePON1 polymorphs 192K (wild-type), 192R and 192Q by rHDL-apoA-I. Delipidated enzymes (0.2 μM) were incubated with increasing concentrations of rHDL-apoA-I, and enzymatic activity was determined with 0.25 mM TBBL. The activity (percentage of stimulation) is presented in relation to the initial activity of the delipidated enzymes (designated as 100%). Data were fitted to the Langmuir saturation curve, from which the activation factor (Vₘₐₓ) and the apparent affinity (Kₐₚₚ) were derived.

FIGS. 4A-B are graphs depicting PON1 inactivation assays in human sera. FIG. 4A is a graph depicting the kinetics of PON1 inactivation in selected human sera. Human sera from healthy individuals were diluted 10-fold in TBS (10 mM Tris pH 8.0, 150 mM NaCl) and subjected to inactivation by 0.25 mM NTA and 1 mM β-mercaptoethanol at 25° C. Residual activity was determined by initial rates of phenyl acetate hydrolysis (2 mM) and plotted as percent of the rate at time zero. Data were fitted either to mono-exponentials (for RR sera), or to double-exponentials (for RQ and QQ sera) curves, from which inactivation rate constants and amplitudes of the phases were derived. Note the large variation in the stability observed mainly with QQ sera. FIG. 4B is a graph depicting correlation between the rate of PON1 inactivation, expressed as the residual activity after 9 hr of incubation with a calcium chelator, and lactonase stimulation, expressed as the ratio of TBBL to DEPCyMC activity, for 54 samples of human sera belonging to the QQ (open squares), RQ (filled triangles) and RR (open circles) genotypes. The crossed lines (+) correspond to the mean values of residual activity and lactonase stimulation for each genotype.

FIG. 5 is a graph depicting the estimated levels of PON1-HDL complex in human sera of 54 healthy individuals. These levels (in arbitrary units) were obtained from the amplitude of the slow phase of inactivation (A₂) (FIG. 1D), multiplied by the levels of DEPCyMC activity (FIG. 1B). Horizontal bars represent the mean value for each group.

FIG. 6A is a graph depicting the correlation between PON1 levels (expressed as DEPCyMC activity) and stability (expressed as percentage residual activity after 9 hrs of inactivation) in 54 human sera of healthy individuals. Correlation factors for linear regression (R)=0.49 for QQ and RQ sera, and 0.54 for RR sera; slopes=0.87, 0.27 and 0.29 for QQ, RQ and RR sera, respectively). DEPCyMC activity (mUnits/ml) was measured at 1 mM, and corresponds to 1 nmol of DEPCyMC hydrolyzed per min per 1 ml undiluted serum. FIG. 6B is a graph depicting the correlation between PON1 levels (expressed as DEPCyMC activity) and lactonase activity (expressed as units TBBL activity) in 54 human sera of healthy individuals. Correlation factors for linear regression (R)=0.75, 0.86 and 0.78 for QQ, RQ and RR sera, respectively; slopes=0.19, 0.26 and 0.21, respectively. TBBLase activity (Units/ml) was measured at 0.25 mM, and corresponds to 1 μmol of TBBL hydrolyzed per min per 1 ml undiluted serum.

FIGS. 7A-B are graphs depicting the correlation between PON1 paraoxonase (FIG. 7A) and phenyl acetate (FIG. 7B) activity, and enzyme levels (expressed as DEPCyMC activity), in 54 human sera of healthy individuals. Correlation factors for linear regression (R)=0.51 and 0.65 for paraoxon and phenyl acetate, respectively; slopes=0.08 and 0.21, respectively. All the activities were measured with 1 mM substrate, and expressed in mUnits/ml for paraoxon and DEPCyMC (1 nmol substrate hydrolyzed per min per 1 ml undiluted serum) or Units/ml for phenyl acetate (1 μmol phenyl acetate hydrolyzed per min per 1 ml undiluted serum).

FIGS. 8A-C are graphs depicting the correlation between the activity levels with paraoxon (FIG. 8A), phenyl acetate (FIG. 8B), and TBBL (FIG. 8C), and levels of PON1-HDL complex (in arbitrary units), in human sera from 54 healthy individuals. The levels of PON1-HDL complex were calculated as described in FIG. 5. All the activities were measured in activity buffer (50 mM Tris pH 8.0, 1 mM CaCl₂) with 1 mM paraoxonase and phenyl acetate, and 0.25 mM TBBL. The activities were expressed in Units/ml for phenyl acetate and TBBL (1 μmol of substrate hydrolyzed per min per 1 ml undiluted serum) and mUnits/ml for paraoxon (1 nmol of substrate hydrolyzed per min per 1 ml undiluted serum). Pearson correlation coefficients for linear regression (R) are 0.64 for paraoxon (FIG. 8A), 0.62 for phenyl acetate (FIG. 8B), and 0.80 for TBBL (FIG. 8C).

### DESCRIPTION OF THE PREFERRED EMBODIMENTS

The present invention is of methods and kits for determining total PON1 levels in a subject which can be used as an aid for diagnosing lipid related disorders.

The principles and operation of the methods according to the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Serum paraoxonase (PON1) hydrolyzes a broad range of substrates, and has been traditionally described as paraoxonase/arylesterase. However, it recently became apparent that PON1 is in fact a lactonase with lipophylic lactones comprising its primary substrates [Khersonsky, O. and D. S. Tawfik, Biochemistry, 2005. 44(16): p. 6371-82]. HDL particles carrying apolipoprotein A-I (apoA-I) bind PON1 with high affinity (nM), dramatically stabilizing the enzyme and stimulating its lipo-lactonase activity [Gaidukov, L. and D. S. Tawfik, Biochemistry, 2005. 44(35): p. 11843-11854].

It has been shown that impairing the lactonase activity of PON1, through mutations of its catalytic dyad, diminishes PON1's ability to prevent LDL oxidation and stimulate macrophage cholesterol efflux, indicating that the anti-atherogenic functions of PON1 are likely to be mediated by its lipo-lactonase activity [Khersonsky, O. and D. S. Tawfik, J Biol Chem, 2006; Rosenblat, M., et al., J Biol Chem, 2006].

To date, blood tests measure phosphotriesterase and arylesterase activities to examine PON1's levels and activity. Because these promiscuous activities of PON1 are hardly, stimulated by HDL, and obviously bear no physiological relevance, these tests cannot predict the levels of the PON1-HDL complex, nor its antiatherogenic potential. In addition, these activities are affected by the 192R/Q polymorphism, and thus can only provide a measure of PON1's total levels within the same genotype. While the paraoxonase activity differs significantly between the R/Q polymorphs the aryl esterase activity of the R polymorph undergoes a 2-fold higher level of catalytic stimulation by HD.

The present inventors developed new sera tests that examine PON1 in light of it being a lipo-lactonase. However, until presently no precise measure of total PON1 levels irrespective of its genotype and HDL status could be determined so that normalization of the enzyme's activity could not be accurately measured. Thus, it was not possible to determine whether variations in lactonase activity result from the differences in enzyme levels, or in the levels of catalytic stimulation by HDL.

Whilst reducing the present invention to practice, the present inventors screened a large number of potential compounds and identified PON1 substrates that may by used to detect total PON1 levels, irrespective of its HDL status and R/Q polymorphism, as well as the degree of catalytic stimulation that follows PON1's binding to HDL-apoA-I.

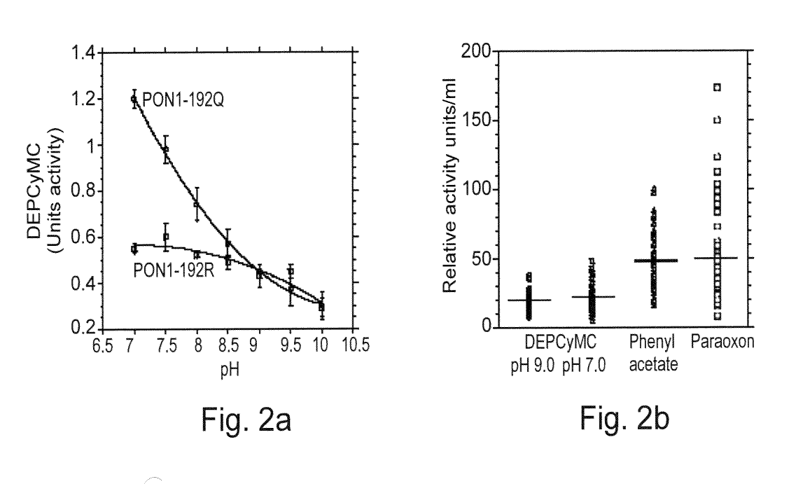

The present inventors showed that the two polymorphs of PON1 comprise an enzymatic activity towards one such substrate, DEPCyMC, which is pH-dependent (FIG. 2A). At pH 7.0, PON1-Q hydrolyzes DEPCyMC with a 2-fold higher activity, but the activity of both polymorphs becomes identical at pH 9.0. Thus it was shown that DEPCyMC at pH 9.0 showed the least variable distribution in tested samples as compared with phenyl acetate, and paraoxon (FIG. 2B).

Thus, according to one aspect of the present invention there is provided a method of determining an amount of total PON1 in a sample of a subject, the method comprising:

(a) contacting the sample with a compound being capable of generating at least one spectrophotometrically detectable moiety upon contact with PON1, under conditions wherein the generating is not dependent on a PON1 status; and

(b) spectrophotometrically measuring a level of the moiety.

The term “PON1” as used herein, refers to mammalian PON1, preferably human (GenBank Accession No. NP 000437.3).

As used herein, the phrase “total PON1” refers to an amount of enzymatically active PON1.

As used herein, the phrase “PON1 status” refers to the genotype of PON1 (such as 192R/Q polymorphism), the environment of PON1 (e.g., circulating or not) and the amount of PON1 bound to other molecules such as HDL.

Preferably, PON1 is present in biological samples derived from an animal subject (e.g., human), such as further described herein below. Preferred sample volumes are between about 10 μl-1 ml.

Exemplary compounds capable of measuring non-status dependent PON1 include but are not limited to 7-O-diethyl phosphoryl 3-cyano 4-methyl 7-hydroxycoumarin (DEPCyMC), 7-O-Diethyl phosphoryl 3-cyano-7-DDAO (DEPDDAO) and 7-O-Diethyl phosphoryl 3-cyano-7-hydroxycoumarin (DEPCyC).

A method of generating DEPCyMC is described in General Materials and Methods herein below.

A method of synthesizing DEPCyC is described herein below:

Triethylamine (0.6 ml, 4.3 mmol) is added to a suspension of 3-cyano-7-hydroxycoumarin (Indofine, N.J.; 562 mg, 3 mmol) in dichloromethane (50 ml) containing diethylphosphorochloridate (0.61 ml, 4.2 mmol). The mixture is stirred for 3 h at room temperature, by which time the insoluble 3-cyano-7-hydroxycoumarin almost completely disappears. TLC on silica (solvent: 5% methanol in dichloromethane) indicates the disappearance of the fluorescent starting material (Rf<0.1) and a non-fluorescent product with Rf≈0.7. The reaction mixture is diluted with dichloromethane (100 ml) and extracted twice with 0.5N HCl, once with 0.1M NaHCO₃ and finally with brine (saturated NaCl) and acidified with HCl. The reaction mixture is dried over Na₂SO₄, the organic solvent evaporated, and the product purified by chromatography on silica using the same solvent system as for TLC. Recrystallization in dichloromethane-ether gave a white crystalline solid (650 mg; 68% yield). ¹H NMR (CDCl₃): 8.22 (s, 1H), 7.57 (d, J=8 Hz, 1H), 7.27 (m, 1H), 7.25 (d, J=5.5 Hz), 4.28 (m, 4H), 1.36 (m, 6H).

A method of synthesizing DEPDDAO is described herein below:

DDAO (49 mg, 0.16 mmol) was dissolved in dichloromethane (30 ml). Diethyl phosphorochloridate (28 μl, 0.19 mmol) was added, followed by triethylamine (27 μl, 0.19 mmol), and the mixture was stirred over night at room temperature. The reaction mixture was washed with HCl at pH˜1 (1×50 ml), brine (1×50 ml), and dried over Na₂SO₄. The organic solvent was evaporated, and the product was purified by chromatography on silica (5% methanol in dichloromethane). Yield: 19 mg, 26.7%

The phrase “spectrophotometrically detectable” as used in the context of the present invention describes a physical phenomena pertaining to the behavior of measurable electromagnetic radiation that has a wavelength in the range from ultraviolet to infrared. Non-limiting examples of spectrophotometrically detectable properties which can be measured quantitatively are color, illuminance and infrared and/or UV specific signature of a chemical compound.

The phrase “spectrophotometrically detectable moiety” therefore describes a moiety, which is formed during an enzymatic assay, and which is characterized by one or more spectrophotometrically detectable properties, as defined herein above. The concentration of such a moiety, which correlates to the enzymatic activity, can thus be quantitatively determined during an enzymatic reaction assay.

The consumption of the compound of the present invention and/or the formation of the product can be measured by following changes in the concentrations of the spectrophotometrically detectable moiety that is formed during the enzymatic catalysis. Examples of spectrophotometric assays include, without limitation, phosphorescence assays, fluorescence assays, chromogenic assays, luminescence assays and illuminiscence assays.

Phosphorescence assays monitor changes in the luminescence produced by a spectrophotometrically detectable moiety after absorbing radiant energy or other types of energy. Phosphorescence is distinguished from fluorescence in that it continues even after the radiation causing it has ceased.

Fluorescence assays monitor changes in the luminescence produced by a spectrophotometrically detectable moiety under stimulation or excitation by light or other forms of electromagnetic radiation or by other means. The light is given off only while the stimulation continues; in this the phenomenon differs from phosphorescence, in which light continues to be emitted after the excitation by other radiation has ceased.

Chromogenic assays monitor changes in color of the assay medium produced by a spectrophotometrically detectable moiety which has a characteristic wavelength.

Luminescence assays monitor changes in the luminescence produced a chemiluminescent and therefore spectrophotometrically detectable moiety generated or consumed during the enzymatic reaction. Luminescence is caused by the movement of electrons within a substance from more energetic states to less energetic states.

Thus according to one embodiment, the rate of DEPCyMC or DEPCyC hydrolysis (1 mM) can be measured by monitoring the absorbance at 400 nm in a final volume of, for example, 200 μl, in Tris-HCl or bis-trispropane buffers at pH 9.0 (ε=22,240 OD/M). Alternatively, the rate of DEPCyMC hydrolysis can be measured by monitoring the fluorescence emission at 450 nm with the excitation set at 400 nm. The rate of DEPDDAO hydrolysis (1 mM) can be measured by monitoring the absorbance at 646 nm in a final volume of, for example, 200 μl, in Tris-HCl buffer at pH 8.0 (ε=18,790 OD/M).

Alternatively, the rate of DEPDDAO hydrolysis can be measured by monitoring the fluorescence emission at 659 nm with the excitation set at 580 nm. According to this aspect of the present invention, the amount of substrate is measured under conditions where generation of the spectrophometrically detectable moiety is not dependent on a PON1 status. The present inventors have shown that such conditions include selection of the pH of the environment. Thus, according to a preferred embodiment of this aspect of the present invention, the measuring of DEPCyMC hydrolysis is performed at pH 9.0, and of DEPDDAO at pH 8.0.

It will be appreciated that measurement of total PON1 may be used in conjunction with the measurement of specific PON1 enzymatic activities in order to determine normalized amounts of PON1 enzymatic activities.

As used herein the phrase “normalized PON1 activity” refers to a specific catalytic activity of the promiscuous catalytic activities of PON1 as a function of total PON1 as described above.

Thus, for example a normalized PON1 esterase activity may be determined by determining PON1's enzymatic activity towards an ester (e.g. naphtyl, benzyl acetate and lipids) and dividing this activity by total PON1.

A normalized PON1 arylesterase activity may be determined by determining PON1's enzymatic activity towards an ester (e.g. phenyl acetate) and dividing this activity by total PON1.

A normalized PON1 phosphotriesterase activity may be determined by determining PON1's enzymatic activity towards a phosphotriester (e.g. paraoxon,) and dividing this activity by total PON1.

A normalized PON1 lactonase activity may be determined by determining PON1's enzymatic activity towards a lactone (e.g. TBBL, δ-valerolactone and γ-dodecanoic lactone) and dividing this activity by total PON1.

According to a preferred embodiment, measurement of lactonase activity is effected using a substrate comprising a lactone, that is capable of generating at least one spectrophotometrically detectable moiety upon hydrolysis thereof.

As is well known in the art, the term “lactone” describes a cyclic carboxylic moiety such as a cyclic ester, which is typically the condensation product of an intramolecular reaction between an alcohol and a carboxylic ester. The latter is oftentimes referred to in the art as “oxo-lactone”. The term “lactone” also typically refers to cyclic thiocarboxylic moieties, and thus include also condensation products of an intramolecular reactions between a thiol group and a carboxylic acid, an alcohol and a thiocarboxylic acid and a thiol group and a thiocarboxylic acid. Such lactones are oftentimes collectively referred to in the art as “thiolactones”.

According to a preferred embodiment of this aspect of the present invention, the substrate used to measure lactonase activity is 5-thiobutyl butyrolactone (TBBL).

It should be noted that the above-described agents for determining total PON1 levels may be included in kits for diagnosing disorders or conditions associated with abnormal levels or activity of PON1 enzyme in a subject.

Examples of disorders associated with abnormal levels or activities of PON1 include, but are not limited to cardiovascular disorders [Costa et al. (2005); Mackness et al. (2004) The role of paraoxonase 1 activity in cardiovascular disease: potential for therapeutic intervention. Am J Cardiovasc Drugs. 2004; 4(4):211-7; Durrington et al (2001) Paraoxonase and atherosclerosis. Arterioscler Thromb Vasc Biol. 2001 21(4):473-80]; pancreatic disorders and neurological disorders. Examples of cardiovascular disorders include but are not limited to atherosclerosis, coronary heart disease, myocardial infarction, peripheral vascular diseases, venous thromboembolism, stroke and pulmonary embolism.

Other examples disorders that are associated with an altered PON1 activity include insulin-dependent (type I) and non-insulin-dependent (type II) diabetes and Alzheimer's disease (Dantoine et al. 2002 Paraoxonase 1 activity: a new vascular marker of dementia? Ann N Y Acad. Sci. 2002 November; 977:96-101). Decreased PON activity has also been found in patients with chronic renal failure, rheumatoid arthritis or Fish-Eye disease (characterized by severe corneal opacities). Hyperthyroidism is also associated with lower serum PON activity, liver diseases, Alzheimer's disease, and vascular dementia. Lower PON activity is also observed in infectious diseases (e.g., during acute phase response). Abnormally low PON levels are also associated with exposure to various exogenous compounds such as environmental chemicals (e.g., metals such as, cobalt, cadmium, nickel, zinc, copper, barium, lanthanum, mercurials; dichloroacetic acid, carbon tetrachloride), drugs (e.g., cholinergic muscarinic antagonist, pravastatin, simvastatin, fluvastatin, alcohol). As mentioned reduced PON levels is also a characteristic of various physiological conditions such as pregnancy, and old age and may be indicative of a subject general health states. For example, smokers exhibit low serum PON1 activity and physical exercise is known to restore PON1 levels in smokers.

As used herein, the term “diagnosing” refers to classifying a disease or a symptom as a PON1 related disorder, determining a predisposition to a PON1 related disorder, determining a severity of a PON1 disorder, monitoring disease progression, forecasting an outcome of a disease and/or prospects of recovery.

The kit may also include other agents (and instructions) for determining the stability of a serum PON1: HDL apoA-I complex. Measurement of complex stability in combination with total serum PON1 levels affords the investigator a gauge as to the amount of PON1-HDL.

The term “PON1:HDL-apoA-I” refers to a complex between PON1 and apoA-I carrying HDL particles.

Exemplary agents for determining the stability of PON1:HDL-apoA-I include PON-1 protein inactivators (e.g. NTA, EDTA and β-mercaptoethanol) and a PON1 substrate (e.g. phenyl acetate). The kit may also comprise β-mercaptoethanol to add to the samples to avoid oxidation.

According to another embodiment of this aspect of the present invention, the kit may comprise agents (and instructions) for determining lactonase activity of PON1. Measurement of lactonase activity in combination with total serum PON1 levels affords the investigator a gauge as to the normalized lactonase activity of a subject.

Thus, the kit may also comprise substrates for PON1 which are capable of measuring the lactonase activity of PON1 such as TBBL.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorpotaed by reference as if fully set forth herein. Other general references are provided throughout this document. The procedures therein are believed to be well known in the art and are provided for the convenience of the reader. All the information contained therein is incorporated herein by reference.

### General Materials and Methods

Lactonase Activity in Sera: Human sera were kindly provided by Michael Aviram. The samples were collected from 54 healthy individuals at Rambam Medical Center (Haifa, Israel) with the approval of the institute's Helsinki committee. Phenotyping sera for the PON1-192R/Q polymorphism was performed by a two-substrate method [Eckerson, H. W., et al., Am J Hum Genet. 35: 1126-1138] as previously described [Gaidukov, L., M. Rosenblat, M. Aviram, and D. S. Tawfik. 2006. J Lipid Res 47: 2492-2502]. Sera were divided into aliquots, supplemented with β-mercaptoethanol (5 mM) to prevent oxidation, and stored frozen at −20° C. All assays were performed in 96-well plates (Nunc), using an automated microplate reader (Bio-Tek; optical length ˜0.5 cm). Lactonase activity was measured in activity buffer (50 mM Tris pH 8.0, 1 mM CaCl₂) containing 0.25 mM of 5-thiobutyl butyrolactone (TBBL) [Gaidukov, L., et al., 2006 J Lipid Res 47: 2492-2502] and 0.5 mM 5,5′-dithio-bis-2-nitrobenzoic acid (DTNB) by monitoring the absorbance at 412 nm in a final volume of 200 μl (ε=7,000 OD/M). The serum was diluted 400-fold in 100 μl of activity buffer complemented with 1 mM DTNB. DTNB was used from 100 mM stock in DMSO. TBBL was used from 250 mM stock in acetonitrile. TBBL was diluted 500-fold in activity buffer containing 2% acetonitrile. The reaction was initiated by adding 100 μl of TBBL (0.5 mM) to 100 μl of sera dilution. The final sera dilution was 800-fold. All the reaction mixtures contained a final 1% acetonitrile. Rates of spontaneous hydrolysis of TBBL in buffer were subtracted from all the measurements. Activities were expressed as U/ml (1 unit=1 μmol of TBBL hydrolyzed per minute per 1 ml of undiluted serum).

Measurements of PON1 Levels in Sera: Total PON1 levels in human sera were assessed by measuring the activity with 7-O-diethyl phosphoryl 3-cyano 4-methyl 7-hydroxycoumarin (DEPCyMC), synthesized as follows: 3-cyano 4-methyl 7-hydroxycoumarin (604 mg, 3 mmol) was dispersed in dichloromethane (50 ml). Diethyl phosphorochloridate (0.61 ml, 4.2 mmol) was added, followed by triethylamine (0.6 ml, 4.3 mmol), and the mixture was stirred over night at room temperature. The reaction mixture was washed with HCl at pH˜1 (2×50 ml), brine (1×50 ml), and dried over Na₂SO₄. The organic solvent was evaporated, and the product was purified by chromatography on silica (2% methanol in dichloromethane). Recrystallization from dichloromethane/ether gave a yellowish solid (410 mg, 40.5% yield). ¹H NMR (250 MHz, CDCl₃) δ (ppm): 7.71-7.74 (d, 1H), 7.31-7.35 (d, 1H), 7.27 (s, 1H), 4.21-4.30 (m, 4H), 2.77 (s, 3H), 1.36-1.42 (m, 6H). ³¹P NMR (250 MHz, CDCl₃) δ (ppm): 6.00 (s). ESI-MS: m/z: 336 [M−1]⁻

For the enzymatic measurements, DEPCyMC was used from 100 mM stock in DMSO, and all the reaction mixtures contained a final 1% DMSO. The activity was measured with 10 μl of serum and 1 mM substrate in 50 mM bis-trispropane, pH 9.0, with 1 mM CaCl₂, by monitoring the absorbance at 400 nm in a final volume of 200 μl (ε=22,240 OD/M). Activities were expressed as mU/ml (1 milli unit=1 nmol of DEPCyMC hydrolyzed per minute per 1 ml of undiluted serum). The normalized lactonase activity was calculated by dividing TBBLase activity of each sample by its DEPCyMC activity.

The DEPCyMC activity of human PON1-192R and Q polymorphs: Purified human PON1-R and Q polymorphs were kindly provided by Michael Aviram. DEPCyMC hydrolysis at 1 mM was measured at the pH range of 7-10 in 50 mM buffers (Tris, bis-trispropane, and CAPS) containing 1 mM CaCl₂. Protein concentrations were verified by activity measurements with phenyl acetate, using the reported specific activities of the R and Q polymorphs [Billecke, S., D. Draganov, R. Counsell, P. Stetson, C. Watson, C. Hsu, and B. N. La Du. 2000. Human serum paraoxonase (PON1) isozymes Q and R hydrolyze lactones and cyclic carbonate esters. Drug Metab Dispos 28: 1335-1342]. The extinction coefficients of the 3-cyano 4-methyl 7-hydroxycoumarin product at different pHs were determined spectrophotometrically. Specific activities were expressed in units (1 unit=1 μmol of DEPCyMC hydrolyzed per minute per 1 mg of protein).

Inhibition of TBBL and DEPCyMC activity in sera: Inhibition of TBBL and DEPCyMC activity in sera samples was measured with EDTA (5 mM), and 2-hydroxyquinoline (0.1 mM), as described [Khersonsky, O., and D. S. Tawfik. 2006. Chromogenic and fluorogenic assays for the lactonase activity of serum paraoxonases. Chembiochem 7: 49-5].

Paraoxonase and aryl esterase activity in sera: Paraoxonase activity in sera samples was measured in activity buffer with 1 mM paraoxon by monitoring the absorbance at 405 nm in a final volume of 200 μl (ε=10,515 OD/M). Arylesterase activity was measured in activity buffer with 1 mM phenyl acetate by monitoring the absorbance at 270 nm in a final volume of 200 μL (ε=7000D/M). Activities were expressed as mU/ml for paraoxon and U/ml for phenyl acetate (1 nmol of paraoxon or 1 μmol of phenyl acetate hydrolyzed per minute per 1 ml of undiluted serum).

Sera Inactivation Assays: Sera inactivation assays were performed as described [Gaidukov, L., M. et al., 2006 J Lipid Res 47: 2492-2502]. Briefly, sera were diluted 10-fold in TBS (10 mM Tris pH 8.0, 150 mM NaCl), and inactivation was initiated by adding an equal volume of inactivation buffer (TBS supplemented with 0.5 mM nitrilotriacetic acid (NTA) and 2 mM β-mercaptoethanol) at 25° C. Residual activity at various time points was determined with 2 mM phenyl acetate in activity buffer. Inactivation rates were fitted well to a mono-exponential or a double-exponential fit. It should be noted that, the reproducibility of these inactivation assays was low, and the inactivation rates varied from one assay to another. It appears that the sera inactivation kinetics are very sensitive to oxidation. Indeed, supplementing sera with β-mercaptoethanol (5 mM) immediately after defrosting, and storing the β-mercaptoethanol supplemented sera at 4° C. for 12-24 hrs before the experiment, yielded more reproducible results.

Estimation of PON1-HDL Levels: PON1-HDL levels in sera were derived from the inactivation assay and the measurements of the total PON1 concentrations with DEPCyMC. For each serum sample, the amplitude of the slow phase of inactivation, A₂ (that corresponds to the fraction of PON1 tightly bound to HDL (19, 27)), was multiplied by the units of DEPCyMC activity (that corresponds to the total concentration of PON1).

Stimulation of TBBL Activity by reconstituted HDL (rHDL): Recombinant PON1 (rePON1) polymorphs were incubated with a range of rHDL-apoA-I concentrations as previously described [Gaidukov, L., M. et al., 2006. J Lipid Res 47: 2492-2502]. TBBLase activity was determined in activity buffer with 0.25 mM TBBL and 0.5 mM DTNB.

### Example 1

### Lipo-Lactonase Activity in Human Sera

Following the observations that the lactonase is the native activity of serum paraoxonases and mediates the antiatherogenic functions of PON1, the lactonase activity in human sera of 54 healthy individuals of the QQ, RQ and RR PON1-192 genotypes was tested using TBBL.

Results

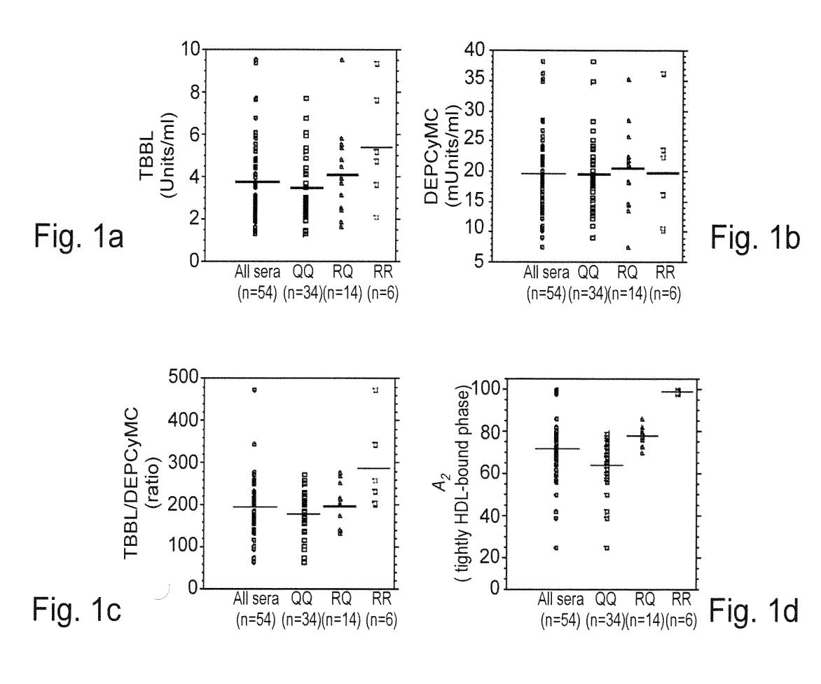

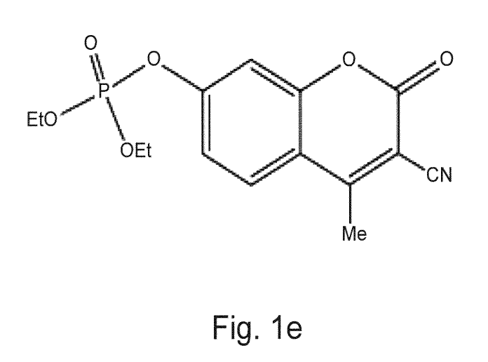

The distribution of the TBBLase activity in 54 human sera is shown in FIG. 1A and Table 1, hereinbelow. Overall, the TBBLase activity varied 7-fold in this sample. The mean lactonase activity in RR sera was found to be 1.5-fold higher than in QQ sera (5.4 units/ml, on average, vs 3.5 units/ml; FIG. 1A, Table 1), in agreement with the observation that PON1-R lactonase activity is better stimulated by HDL than PON1-Q [Gaidukov, L., et al., 2006, J Lipid Res 47: 2492-2502]. Table 1 herein below, summarizes the lactonase activity, PON1 levels, lactonase stimulation and PON1-HDL levels, in human sera of 54 healthy individuals.

TABLE 1

| | | | TBBL/ | PON1-HDL^{d} |
|---|---|---|---|---|
| | TBBL^{a} | DEPCyMC^{b} | DEPCyMC^{c} | (arbitrary |
| | (Units/ml) | (mUnits/ml) | (ratio) | units) |
| All sera | 3.8 ± 1.9 | 19.7 ± 6.7 | 194.3 ± 65.6 | 14.3 ± 6.2 |
| (n = 54) | | | | |
| QQ sera | 3.5 ± 1.6 | 19.5 ± 6.3 | 177.4 ± 49.9 | 12.8 ± 5.2 |
| (n = 34) | | | | |
| RQ sera | 4.1 ± 2.1 | 20.4 ± 6.9 | 196.4 ± 48.5 | 15.8 ± 5.4 |
| (n = 14) | | | | |
| RR sera | 5.4 ± 2.7 | 19.8 ± 9.8 | 285.4 ± 105.5 | 19.6 ± 9.6 |
| (n = 6) | | | | |

^{a}Lactonase activity was measured with TBBL (0.25 mM), and expressed as μmol of TBBL hydrolyzed per min per 1 ml undiluted serum.
^{b}PON1 levels were measured with DEPCyMC (1 mM) at pH 9.0, and expressed as nmol of DEPCyMC hydrolyzed per min per 1 ml undiluted serum.
^{c}Lactonase stimulation was calculated as the ratio of TBBL to DEPCyMC activity for each individual serum.
^{d}PON1-HDL levels were obtained by multiplying, for each serum sample, the amplitude of the slow phase of inactivation which corresponds to the fraction of tightly HDL-bound PON1 (A₂ values; FIG. 1D), by the level of its DEPCyMC activity (FIG. 1B) which corresponds to the total concentration of serum PON1.
All the numbers represent mean values and S.D. for each 192R/Q genotype.

### Example 2

### Total PON1 Levels in Human Sera

The differences in TBBLase activity are the combined outcome of two factors: differences in the absolute concentrations of PON1, and different levels of stimulation in each serum. To separate these factors, the present inventors searched for a substrate that would have exactly the same specific activity with PON1-R and Q polymorphs, and would not undergo any stimulation by HDL, and thus would reflect the total levels of the PON1 protein in sera, similarly to the previously described PON1 ELISA [Blatter Garin, M. C., C. et al. 1994. Biochem J 304 (Pt 2): 549-554]. Phenyl acetate that is usually used as a surrogate marker for PON1 concentration is not a suitable substrate since it undergoes ca. 2-fold stimulation upon HDL binding [Gaidukov, L., M. et al., 2006, J Lipid Res 47: 2492-2502]. Paraoxonase activity, although unaffected by HDL binding, differs between the R and Q polymorphs.

### Results

A large number of potential substrates were screened and a new chromogenic/fluorogenic phosphotriester DEPCyMC (FIG. 1E) that is ideal for measuring total PON1 concentrations was identified. It was found that, similarly to other phosphotriesters, DEPCyMC is not stimulated by HDL binding. The DEPCyMC activity of the two polymorphs is pH-dependent, and can thus be tuned (FIG. 2A). At pH 7.0 PON1-Q hydrolyzes DEPCyMC with a 2-fold higher activity, but the activity of both polymorphs becomes identical at pH 9.0. Both TBBL and DEPCyMC activities in sera appear to be highly specific to PON1, since both are efficiently inhibited (≧92%) by the calcium chelator EDTA (5 mM), and the selective competitive inhibitor of PON12-hydroxyquinoline (0.1 mM).

It was therefore surmised that DEPCyMC activity can provide a reliable measure of the total PON1 concentration in sera regardless of its polymorphism and HDL status. This was further supported by comparing the measurements of PON1 activities in sera with DEPCyMC (at pH 7 and 9), phenyl acetate, and paraoxon (FIG. 2B and Table 2, hereinbelow). Table 2 summarizes the distribution of activity with DEPCyMC, phenyl acetate and paraoxon in human sera from 54 healthy individuals.

TABLE 2

| | Range | Mean ± S.D. |
|---|---|---|
| | (Units activity) | (Units activity) |
| DEPCyMC | 7.5-38.2 | 19.7 ± 6.7 |
| (pH 9.0) | (5) | |
| DEPCyMC | 5.0-48.1 | 21.7 ± 9.7 |
| (pH 7.0) | (10) | |
| Phenyl acetate | 15.6-100.6 | 48.2 ± 20.9 |
| | (6) | |
| Paraoxon | 7.7-173.5 | 49.5 ± 36.6 |
| | (23) | |

DEPCyMC at pH 9.0 showed the least variable distribution (as revealed by the lowest standard deviation around the mean activity): the 54 sera samples exhibited variations in PON1 activities in the range of 5-fold (FIG. 2B; Table 2, herein above). The mean DEPCyMC activity at pH 9 was also found to be essentially identical for the R and Q genotypes (FIG. 1B and Table 1 herein above). As expected, DEPCyMC hydrolysis at pH 7.0 exhibited higher mean activity and much larger variations between the sera samples (in the range of 10-fold) due to differences in the specific activities of PON1-Q compared to PON1-R (FIG. 2A). The aryl esterase and paraoxonase activities also revealed large variations (6-23 fold) between the samples, with large standard deviations around the mean activity and differences between R and Q genotypes (FIG. 2B; Table 2). These large variations between individuals result not only from differences in total PON1 levels, but also from differences in the R and Q genotype (DEPCyMC at pH 7, and paraoxon), and differences in the degree of HDL stimulation (phenyl acetate). In contrast, the 5-fold variations in DEPCyMC at pH 9 reflect differences only in total enzyme concentration, as previously observed in measures of PON1 protein by ELISA [Blatter Garin, M. C., C. Abbott, et al., 1994, Biochem J 304 (Pt 2):549-554].

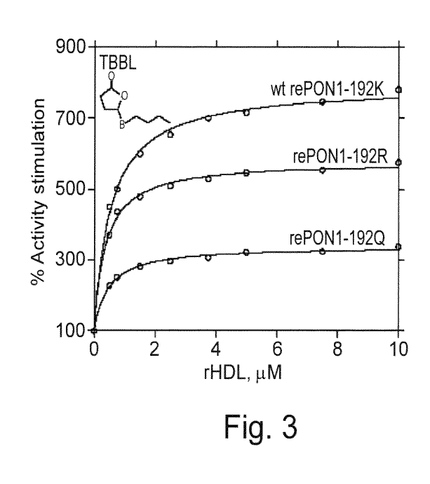

The ratio of TBBL to DEPCyMC activity provides the normalized lactonase activity, and therefore corresponds to the degree of HDL stimulation. In absence of HDL, purified human PON1 polymorphs exhibit the same specific activity for TBBL (1.0±0.1 μmol/min/mg of protein at 0.25 mM TBBL), and thus the differences in the lactonase activity between individual sera result solely from differences in the lactonase stimulation by HDL. In the in vitro system of rePON1 or human PON1 R/Q polymorphs, and rHDL, the lipo-lactonase activities differed by a factor of ca. 2-fold in the degree of stimulation by HDL-apoA-I (FIG. 3). In agreement with these observations, the RR sera exhibit 1.6-fold higher mean normalized lactonase activity than the QQ sera (FIG. 1C and Table 1, hereinabove).

### Example 3

### Inactivation Assays of PON1 in Human Sera

It has been shown that determining the rate of PON1's chelator-mediated inactivation provides a measure of the level of tightly vs. loosely HDL-bound enzyme both in reconstituted in vitro system [Gaidukov, L., and D. S. Tawfik. 2005. Biochemistry 44: 11843-11854], and in sera samples [Gaidukov, L., M. Rosenblat, M. Aviram, and D. S. Tawfik. 2006. J Lipid Res 47: 2492-2502].

The human sera was subjected to inactivation by a low affinity calcium chelator nitrilotriacetic acid (NTA) that chelates PON1's essential calcium ions, and the reducing agent β-mercaptoethanol. The rate of inactivation was monitored by measuring the residual arylesterase activity at different time points, and comparing it to the initial activity.

Results

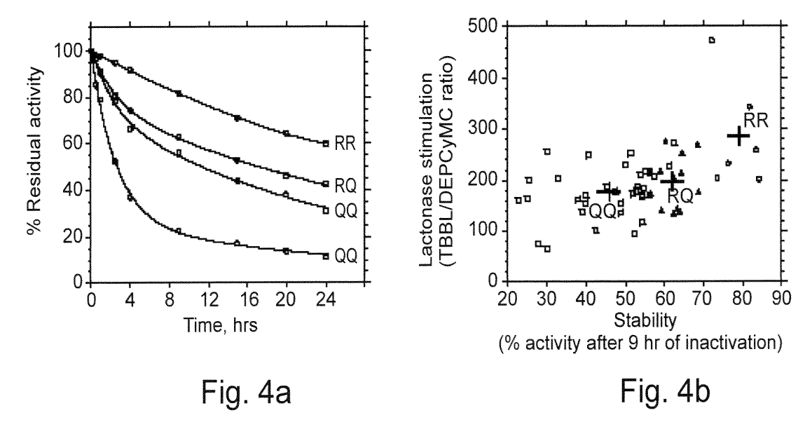

The results of inactivation profiles of representative sera are depicted in FIG. 4A.

PON1's inactivation rates differed markedly between different sera samples. Inactivation kinetics followed either a mono-exponential slow rate decay (e.g., FIG. 4A, RR sample), or a double-exponential regime in which a first (fast) inactivation phase was followed by a second (slow) phase (RQ and QQ samples). The stable phase corresponds to PON1 that is tightly, or effectively, bound to HDL, while the unstable phase corresponds to the “loosely-bound” PON1 population. Thus, by following PON1's inactivation in sera, the fractions of the tightly and loosely HDL-bound PON1 population can be derived (A₂ and A₁, respectively). The mono-exponential decay observed primarily with the homozygotes RR individuals reflects a favorable partitioning of PON1 in the tightly bound phase (i.e. the percentage of the slow inactivation phase, A₂, is 100%). In other sera, where inactivation obeys a double-exponential regime, A₂ ranges from 25 to 86%. The distribution of the tightly HDL-bound fractions derived from the inactivation assay for the 54 sera samples is shown in FIG. 1D.

Notably, the lactonase stimulation measurements (FIG. 1C) and the inactivation rates (FIG. 1D) appear to correlate and cluster in accordance with the 192R/Q genotypes (FIG. 4B).

### Example 4

### Estimation of PON1-HDL Levels

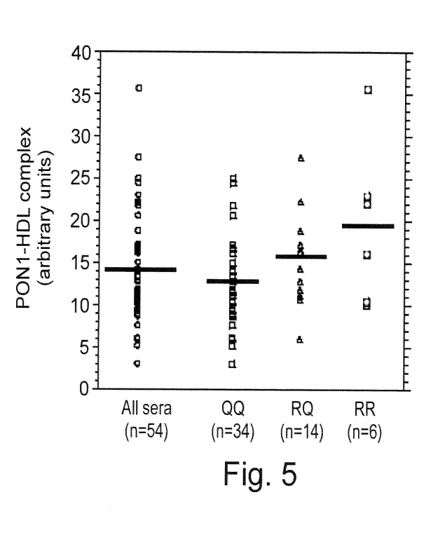

The levels of PON1-HDL complex can be derived by combining the inactivation measurements (FIG. 1D) with the measurements of PON1's total concentration using DEPCyMC (FIG. 1B). The amplitude of the second inactivation phase, A₂, corresponds to the fraction of tightly HDL-bound PON1. Multiplying A₂ by the total PON1 concentration (derived from the DEPCyMC rates), yields the level of PON1 that is tightly, or efficiently, associated with HDL, namely the level of PON1-HDL complex. The derived HDL-associated PON1 levels in 54 human sera are shown in FIG. 5 and Table 1, herein above. These levels vary considerably between individuals (between 3-36 in arbitrary units). The mean HDL-PON1 levels are 1.5-fold higher for the RR sera compared to the QQ sera mainly due to the higher A₂ values. However, there is a large overlap in the estimated PON1-HDL levels between PON1 polymorphs and the differences between the individuals go well beyond the effect of the 192R/Q genotype. Thus, even in the small sample examined here there are many QQ individuals with higher estimated PON1-HDL levels than some RR individuals.

### DISCUSSION

Lactonase activity amongst the tested population varied by ±7-fold in a sample (n=54) of healthy individuals (FIG. 1A). To reveal whether these variations result from the differences in enzyme levels, or in the levels of catalytic stimulation by HDL, a substrate was identified that would not be affected by HDL-binding, nor by the R/Q polymorphism, and thus could be applied to determine PON1's total concentration. At pH 9.0, PON1-192R/Q polymorphs hydrolyze this substrate (DEPCyMC) at the same rate, with no effects of HDL-binding. DEPCyMC activity is specific to PON1, requires small amount of serum (10 μl), and can be assayed in a high-throughput manner by both absorbance and fluorescence.

A large variability (10-40-fold) in PON1 sera activity is observed with many substrates. However, direct measurements of PON1 protein levels by ELISA showed variations of only 5-fold [Blatter Garin, M. C., et al., 1994, Biochem J 304 (Pt 2): 549-554]. This discrepancy results from the fact that most activities reflect not only the differences in total PON1 levels, but also the differences in the specific activity of various genotypes, and/or in the degree of catalytic stimulation by HDL. DEPCyMC activity, on the other hand, solely reflects the enzyme concentrations and can be compared across PON1 genotypes. Thus, similarly to the direct PON1 quantification by ELISA, measurements of DEPCyMC activity showed variations of 5-fold in PON1 concentrations in different sera (FIG. 1B).

The ratio of lipo-lactonase (measured with TBBL) to DEPCyMC activity yields the levels of lactonase stimulation, and indicates clear differences between the three types of sera (FIG. 1C and Table 1). The RR sera exhibit, on average, a 1.6-fold higher stimulation levels than the QQ sera. This difference is in agreement with the levels of stimulation observed with lipo-lactones in vitro [Gaidukov, L., M. et al., 2006, J Lipid Res 47: 2492-2502]. However, the overall differences in lipo-lactonase activity (FIG. 1A) and stimulation (FIG. 1C) of PON1's R/Q genotypes seem to be masked by much larger variations in total enzyme concentrations, as well as other factors such as the degree of HDL-binding.

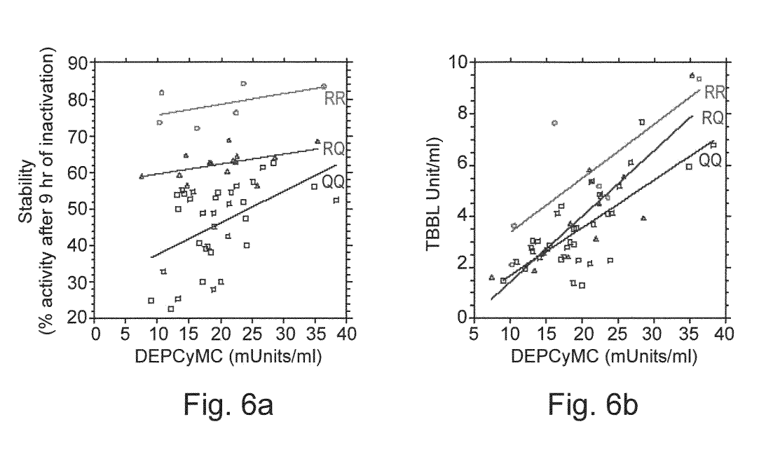

The measure of catalytic stimulation (TBBL/DEPCyMC; FIG. 1C), and the fraction of tightly-bound PON1 from the inactivation assays (FIG. 1D) appear to correlate (FIG. 4B). Thus, as observed in vitro with recombinant PON1 polymorphs and reconstituted HDL [Gaidukov, L., M. et al., 2006, J Lipid Res 47: 2492-250], the tightly HDL-associated fraction of PON1 is more stable and exhibits higher lactonase activity. This fraction may therefore represent the “biologically active” population of PON1, while the loosely bound PON1 is much less stable and largely non active. Interestingly, large heterogeneity is observed in the fraction of tightly bound PON1. This heterogeneity is also related to variations in PON1's concentrations. Thus, it is observed that, individuals with higher serum PON1 concentrations, and in particular Q polymorphs, also exhibit higher stability (slopes for linear regression are 0.87, 0.27 and 0.29 for the QQ, RQ and RR sera, respectively; FIG. 6A) and higher lactonase activity (slopes for linear regression are 0.19, 0.26 and 0.21 for the QQ, RQ and RR sera, respectively; FIG. 6B). This observation might be related to the positive correlation between serum PON1 and HDL levels [Blatter Garin, M. C., et al., 1994, J Lipid Res 47: 515-520; Van Himbergen, T. M., et al, 2005. J Lipid Res 46: 445-451]. Increased PON1 levels therefore seem to shift the binding equilibrium and increase the levels of tightly HDL-bound PON1, thus increasing PON1's stability and lactonase activity.

By determining the percentage of PON1 which is tightly bound to HDL, and the total PON1 concentration, the present inventors were able to assess the levels of HDL-associated PON1 (FIG. 5). The results show that these levels vary between individuals (3-36 arbitrary units; or ˜12-fold; FIG. 5, Table 1, herein above) to much larger degree than total enzyme concentrations (5-fold; FIG. 1B). Although the mean values for the estimated HDL-PON1 complex in human sera are 1.5-fold higher for the RR than the QQ genotypes, the effect of the R/Q polymorphism appears to play a minor role and might result from the small sample size examined here (n=54) with only six RR sera. The intra-genotype variability (4-8 fold) is significantly larger than the mean inter-genotype differences (1.2-1.5 fold) and thus, even in the small sample examined here, many individuals were observed with the inferior QQ genotype that exhibit higher levels of HDL-associated PON1, and subsequently higher lipo-lactonase activity, than most RR individuals. Numerous case-control studies that tried to relate the PON1 R/Q polymorphism with the risk of cardiovascular disease yielded conflicting results, with some studies indicating the RR genotype as a risk factor and others indicating no association between the disease and either allele. The present tests show that PON1 R/Q polymorphism plays a relatively minor role in determining the levels of HDL-PON1 complex, and may explain why previous attempts to correlate 192R/Q phenotype with predisposition for atherosclerosis failed. Similar conclusions were derived from several previous studies that suggested that PON1's phenotype may be more important than its genotype [Mackness, M., and B. Mackness. 2004. Free Radic Biol Med 37: 1317-1323.

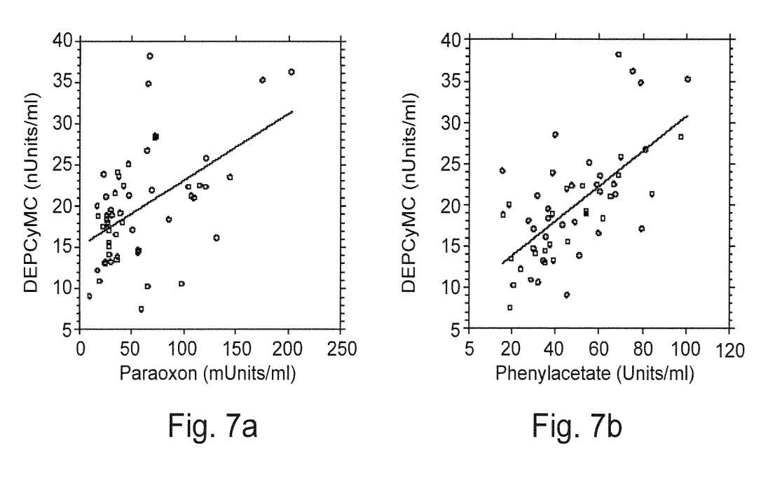

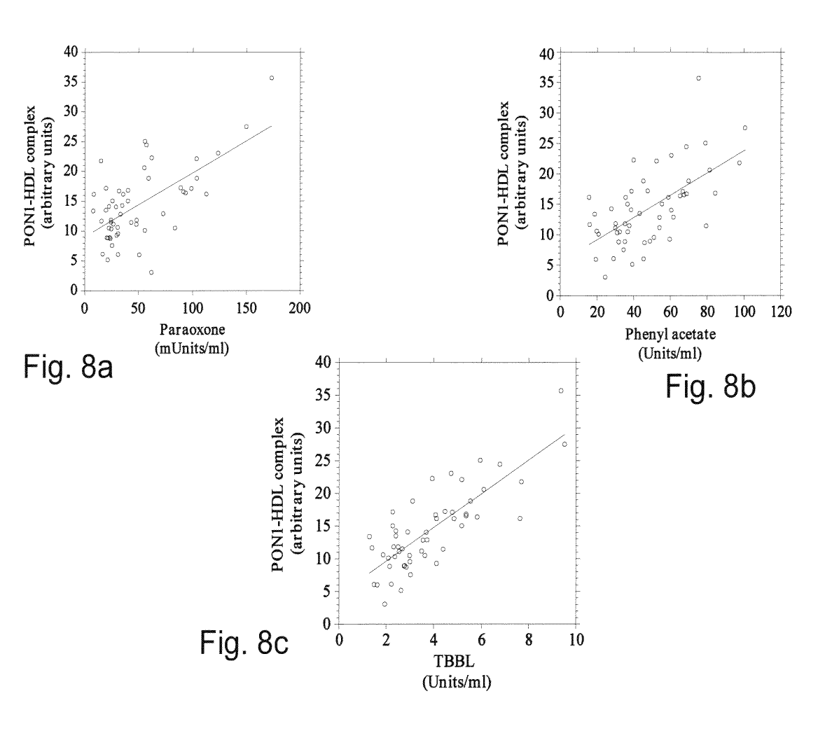

Paraoxonase and aryl esterase activities have been traditionally used to test PON1 levels and activity, and have been suggested as a marker for the prediction of cardiovascular disease. The phosphotriesterase and aryl esterase activity of PON1 are positively correlated with total PON1 concentrations (the Pearson correlation coefficients for linear regression, R, are 0.51 and 0.65 for paraoxon and phenyl acetate, respectively; FIGS. 7A-B), which in turn, are positively correlated with PON1-HDL levels and stability (FIG. 6A). However, the correlation between the paraoxonase and aryl esterase activity and HDL-PON1 levels is quite poor (R=0.64 and 0.62, respectively) (FIGS. 8A-B). Indeed, PON1-HDL levels at around the mean paraoxonase and aryl esterase activity (50 units) vary by as much as 8-fold for paraoxon (between 3-25 arbitrary units) and 5-fold for phenyl acetate (between 5-23 arbitrary units). This may explain why previous attempts to correlate PON1 phosphotriesterase and aryl esterase activities with the risk of atherosclerosis did not yield significant results [Mackness, M., and B. Mackness. 2004. Free Radic Biol Med 37: 1317-1323]. Moreover, it is now know that these activities are promiscuous, non-physiological functions of PON1. In contrast, the lactonase activity is the primary function of PON1 [Khersonsky, O., and D. S. Tawfik. 2005. Biochemistry 44: 6371-6382; Draganov, D. I., et al., 2005. J Lipid Res 46: 1239-1247], is greatly stimulated by HDL, and appears to mediate at least two of PON1's antiatherogenic functions [Rosenblat, M., L. 2006 J Biol Chem 281: 7657-7665]. Indeed, the TBBLase activity exhibits a significantly better correlation with the levels of PON1-HDL (R=0.80) with only 2.6-fold variations around the mean activity (between 9-23 arbitrary units; FIG. 8C).

In conclusion, the new sera tests involve the measurements of PON1's absolute levels, lipo-lactonase activity, and the degree of catalytic stimulation. The minimal test measures the total PON1 protein levels with DEPCyMC, and the lipo-lactonase activity with TBBL. Total TBBLase activity appears to be in reasonable correlation with the levels of HDL-associated PON1 (FIG. 8B), and the normalized TBBLase activity (TBBL/DEPCyMC ratio; FIG. 1C) reflects the efficiency of catalytic stimulation by HDL. A more comprehensive measure may involve the inactivation assay to derive the fraction of tightly-bound PON1 (A₂; FIG. 1D). This fraction is a marker of the degree of HDL binding, and in combination with PON1 DEPCyMC activity reflects the levels of HDL-associated PON1. Taken together, these tests provide the integrative measures of the activity and levels of PON1 and HDL particles onto which it is bound, and is likely to provide a better correlation with the antiatherogenic activity than the current paraoxonase and aryl esterase assays. Future studies may reveal whether these new tests of sera PON1, perhaps in conjunction with other assays that address the levels of various types of HDLs, LDLs, apolipoproteins, and other proteins and factors related to atherosclerosis comprise reliable indicators as well as predictors of atherosclerosis.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A method of determining an amount of total PON1 in a sample of a subject, the method comprising:
(a) contacting the sample with a compound being capable of generating at least one spectrophotometrically detectable moiety upon contact with PON1, under conditions wherein said generating is not dependent on a PON1 status; and
(b) spectrophotometrically measuring a level of said moiety, thereby determining an amount of total PON1 in the sample.

2. A method of determining a normalized enzymatic activity of PON1 in a sample of a subject, the method comprising:
(a) contacting the sample with a compound being capable of generating at least one spectrophotometrically detectable moiety upon contact with PON1, under conditions wherein said generating is not dependent on a PON1 status;
(b) spectrophotometrically measuring a level of said moiety, thereby determining an amount of total PON1 in the sample; and
(c) measuring a PON1 enzymatic activity selected from the group consisting of a lactonase activity, a paraoxonase activity and an aryl esterase activity, wherein a ratio of said PON1 enzymatic activity and said amount of total PON1 is said normalized enzymatic activity of PON1.

3. The compound 7-O-Diethylphosphoryl-(3-cyano 4-methyl 7-hydroxycuomarin).

4. The method of claim 1, wherein said compound is selected from the group consisting of 7-O-Diethyl phosphoryl 3-cyano-7-DDAO, 7-O-diethyl phosphoryl 3-cyano 4-methyl 7-hydroxycoumarin (DEPCyMC) and 7-O-Diethyl phosphoryl 3-cyano-7-hydroxycoumarin (DEPCyC).

5. The method of claim 1, wherein said conditions comprise contacting the sample with said compound at pH 9.

6. The method of claim 2, wherein measuring said lactonase activity is effected by:
(a) contacting the sample with a compound containing at least one lactone, wherein said compound is capable of generating at least one spectrophotometrically detectable moiety upon hydrolysis of said lactone; and
(b) spectrophotometrically measuring a level of said moiety.

7. The method of claim 6, wherein said compound is 5-thiobutyl butyrolactone (TBBL).

8. A kit for diagnosing a disorder associated with abnormal levels or activity of a PON1 in a subject, the kit comprising a phophotriester compound selected from the group consisting of 7-O-Diethyl phosphoryl 3-cyano-7-DDAO, 7-O-diethyl phosphoryl 3-cyano 4-methyl 7-hydroxycoumarin (DEPCyMC) and 7-O-Diethyl phosphoryl 3-cyano-7-hydroxycoumarin (DEPCyC) and instructions for measuring a total amount of PON1.

9. The kit of claim 8, further comprising at least one agent for determining in a sample of a subject stability of a serum PON1:HDL apoA-I complex.

10. The kit of claim 9, wherein said at least one agent for determining a stability of a serum PON1: HDL apoA-I complex is an agent capable of measuring an inactivation rate of an enzymatic activity of a PON1 of said PON1: HDL apoA-I complex.

11. The kit of claim 10, wherein said at least one agent is a PON1 inactivator.

12. The kit of claim 11, wherein said PON1 inactivator is NTA, β-mercaptoethanol or both.

13. The kit of claim 10, wherein said at least one agent is phenyl acetate.

14. The kit of claim 8, further comprising at least one reagent for determining a lactonase activity of serum PON1.

15. The kit of claim 14, wherein said at least one reagent is 5-(thiobutyl)-butyrolactone (TBBL).

16. The kit of claim 8, wherein said disorder associated with abnormal levels or activity of a PON1 is selected from the group consisting of a cardiovascular disorder, a pancreatic disorder and a neurological disorder.

17. The kit of claim 16, wherein said cardiovascular disorder is selected from the group consisting of atherosclerosis, coronary heart disease, myocardial infarction, peripheral vascular diseases, venous thromboembolism and pulmonary embolism.

18. The method of claim 2, wherein said compound is selected from the group consisting of 7-O-Diethyl phosphoryl 3-cyano-7-DDAO, 7-O-diethyl phosphoryl 3-cyano 4-methyl 7-hydroxycoumarin (DEPCyMC) and 7-O-Diethyl phosphoryl 3-cyano-7-hydroxycoumarin (DEPCyC).

19. The method of claim 2, wherein said conditions comprise contacting the sample with said compound at pH 9.
